(19) United States
(12) Patent Application Publication
(10) Pub. No.: US 2020/0297039 A1
(43) Pub. Date: Sep. 24, 2020

(54) PROTECTIVE FLAVORED THONG

(71) Applicants: Jeffrey Dean Parker, Othello, WA (US); Melissa Mae Andrews, Reno, NV (US); Theresa Marie Hernandez, Sacramento, CA (US)

(72) Inventors: Jeffrey Dean Parker, Othello, WA (US); Melissa Mae Andrews, Reno, NV (US); Theresa Marie Hernandez, Sacramento, CA (US)

(21) Appl. No.: 16/361,920

(22) Filed: Mar. 22, 2019

(51) Int. Cl. A41B 9/00; A41B 9/14; A23G 3/56; A61F 6/06; A41B 17/00

(52) U.S. Cl. CPC A41B 9/002; A41B 9/14; A41B 17/00; A61F 6/065; A23G 3/563

(57) ABSTRACT

A protective flavored thong including a waistband configured to extend around the waist of a wearer, a crotch band, and at least one flavor ridge applied centrally along the crotch band. The thong provides an erotic female under-garment that is convenient to wear, visually appealing, accentuates the wearer's womanly curves, and provides protection against sexually transmitted diseases during cunnilingus for both the wearer and her sexual partner. Additionally, the thong includes a flavor ridge that imparts a pleasant flavor, also providing lingual and tactile guidance to the woman's partner during cunnilingus in an environment with possibly limited visual guidance. The flavor ridge can be applied to a backing layer that is applied centrally along the crotch band. The crotch band can include a panel of low-density polyethylene (LDPE) to serve as a protective barrier against transmission of STDs between the wearer and her partner during cunnilingus.

### FIELD OF THE INVENTION

This invention relates generally to female garments, and more particularly to prophylactic garments for enhancing erotic interactions.

### BACKGROUND OF THE INVENTION

As part of an erotic interaction, it is common for female participants to wear under-garments that can enhance a sexual experience. Common examples of female under-garments that provide increased sexual titillation are: bras, panties, thongs, and G-strings.

A thong is an article of female underwear that includes a narrow strip of fabric or other material that passes between a woman's thighs and connects to a waistband.

Women often wear thongs to increase a sense of erotic excitement between themselves and their partners while engaging in sexual activities.

Thongs can increase the woman's attractiveness in several ways. Because a thong is a skimpy and very revealing article of clothing, much of the woman's skin is exposed, enhancing her erotic appeal. In addition, thongs are often tailored to accentuate the curves of a woman's body, and thongs can include feminine decorations such as feminine colors, or lacey fabrics.

Thongs are usually made from materials such as cotton, Rayon®, polyester, and Spandex®. Unfortunately, these materials will allow the passage of bodily fluids containing bacteria and viruses, and therefore typically thongs do not prevent the transmission of sexually transmitted diseases (STDs) such as syphilis and chlamydia (bacterial), and herpes, hepatitis C, and HPV (viral).

An example of a prophylactic undergarment is taught in Wallace, US Patent Application 2006/0042639 A1. Wallace teaches a prophylactic undergarment for use by a female wearer that includes an elastomeric portion that reduces the transmission of STDs. However, the woman's garment of Wallace has disadvantages. The prophylactic garment covers more skin than needed to provide prophylaxis during cunnilingus, thereby reducing the erotic attractiveness of the garment without concomitant benefit. In addition, the garment includes two prophylactic tubes configured to be inserted into the woman's vaginal canal and anus. Therefore the garment is difficult to put on, and may be uncomfortable to wear because of the two intrusive tubes.

### SUMMARY

The protective flavored thong of the invention provides an erotic female under-garment that is convenient to wear, visually appealing, accentuates the wearer's natural womanly curves, and provides protection against transmission of STDs for both the wearer and her sexual partner during oral sex.

In addition, the protective flavored thong of the invention includes a flavor ridge that runs along a central axis of the crotch that provides a pleasant scent and flavor, while also providing lingual and/or tactile guidance to the mouth and/or tongue of the wearer's partner while performing cunnilingus in a dark environment.

The protective flavored thong of the invention can include a panel of low-density polyethylene (LDPE) (similar to Saran Wrap®) that acts as a protective barrier during cunnilingus to protect against transmission of STDs between the wearer and her partner.

Another benefit of the protective flavored thong of the invention is that it is disposable.

A general aspect of the invention is a protective flavored thong that includes: a waistband configured to extend around a waist of a wearer, the waistband having a front portion and a rear portion; a crotch band having a wide end and a narrow end, the narrow end being attached at the rear portion of the waistband, and the wide end being attached along the front portion of the waistband; and at least one flavor ridge applied centrally along the crotch band.

In some embodiments, the waistband includes a band of elastic silicone. In some embodiments, the crotch band includes two bands of elastic silicone, each band of elastic silicone running along an edge of the crotch band.

In some embodiments, the crotch band includes a panel of low-density polyethylene (LDPE).

In some embodiments, the crotch band includes a panel of material that is impervious to fluids.

In some embodiments, the at least one flavor ridge includes at least one of: a flavoring agent, a souring agent, and a sweetening agent.

In some embodiments, the protective flavored thong further includes: a protective strip removably attached along the at least one flavor ridge, the protective strip being configured to protect freshness of the at least one flavor ridge while the at least one protective strip is attached to the at least one flavor ridge, and to be removable so as to leave the at least one flavor ridge substantially in place after the protective strip is removed.

In some embodiments, the protective strip includes a tab portion configured to facilitate removal of the protective strip.

In some embodiments, the at least one flavor ridge includes a pair of flavor ridges symmetrically placed along a central axis of the crotch band. In some embodiments, the protective flavored thong further includes: a backing layer, the backing layer applied centrally along the crotch band, the flavor ridge being applied centrally along the backing layer.

In some embodiments, the backing layer includes a coating of light adhesive that improves the adhesion of the backing layer to the crotch band. In some embodiments, the backing layer includes a smooth front-facing surface that retains its shape so as to facilitate licking of the flavor ridge off of the backing layer.

In some embodiments, the protective flavored thong is packaged in a folded condition.

In some embodiments, the at least one flavor ridge folds once or twice when packaged in the folded condition.

In some embodiments, the at least one flavor ridge is flavored to resemble at least one of: lemon, lime, orange, cherry, pineapple.

### BRIEF DESCRIPTION OF THE DRAWINGS

FIG. 1A is an isometric front view of a protective flavored thong having a waistband, a crotch band having a wide end and a narrow end connected to the front and rear of the waistband, a band of elastic silicone running along each side of the crotch band, and a protective strip to be removed before use, the protective strip covering a flavor ridge running centrally along the crotch band, the protective strip having a tab portion.

FIG. 1B is an isometric rear view of the protective flavored thong of FIG. 1A, showing the rear portion of the protective strip covering the narrow end of the crotch band.

FIG. 2A is an isometric front view of the protective flavored thong of FIG. 1A, showing the protective strip being removed from the wide end of the crotch band by lifting the tab portion of the protective strip to expose the flavor ridge, the flavor ridge being shown in dotted line where covered by the protective strip, and seen through the crotch band.

FIG. 2B is an isometric rear view of the protective flavored thong of FIG. 1A, showing the protective strip being lifted away from the narrow end of the crotch band to further expose the flavor ridge, the flavor ridge being shown in dotted line where covered by the protective strip, and seen through the crotch band.

FIG. 3A is a front view of another embodiment of a protective flavored thong, this embodiment having a backing layer that is applied centrally to the crotch band, the flavor ridge running along the backing layer, this view showing the backing layer and the flavor ridge after the protective strip has been entirely removed.

FIG. 3B is a rear view of the protective flavored thong of FIG. 3A, showing the backing layer and the flavor ridge after the protective strip has been entirely removed.

FIG. 4 is a cross-sectional view of the crotch band, the flavor ridge, and the protective strip as it is being peeled away from the protective flavor ridge.

FIG. 5 is a front view of the protective flavored thong being worn around the waist of a wearer, showing the waistband, two bands of elastic silicone, and one flavor ridge.

### DETAILED DESCRIPTION

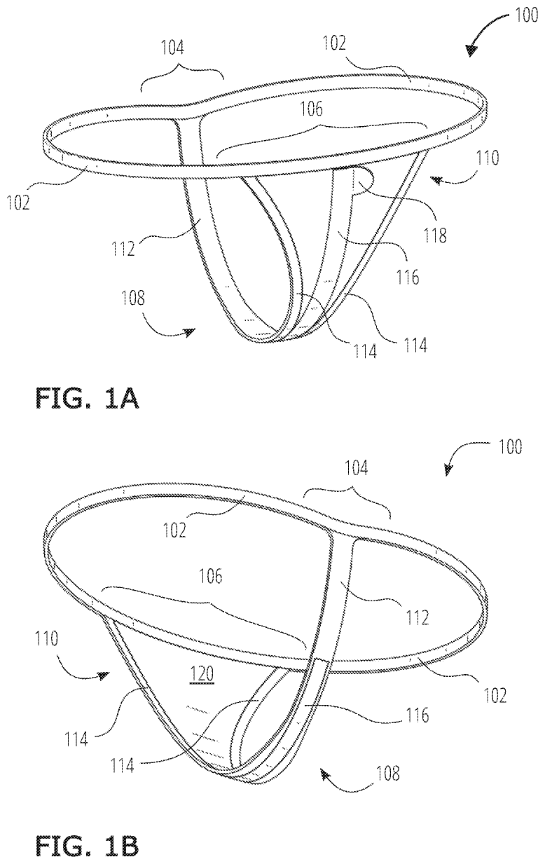

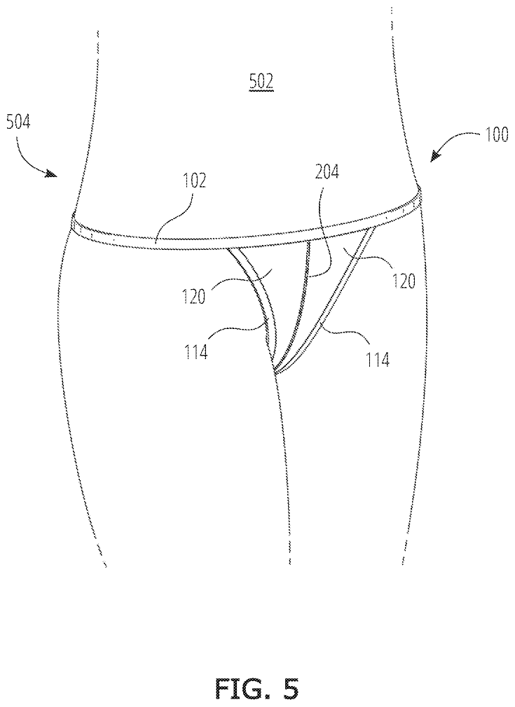

With reference to FIG. 1A, an isometric front view of a protective flavored thong 100 is shown, having a waistband 102 configured to be worn around a waist 504 of a wearer 502 (both shown in FIG. 5). The waistband 102 includes a front portion 106 and a rear portion 104. In some embodiments, the waistband 102 includes a band of elastic silicone being threaded into, woven into, bonded with, or forming a core of the fabric of the waistband 102.

The protective flavored thong 100 also includes a crotch band 108 that includes a wide end 110 and a narrow end 112. In this embodiment, the crotch band 108 includes two bands of elastic silicone 114, each band of elastic silicone 114 running along an edge of the crotch band 108. The two bands of elastic silicone 114 provide structural integrity and also maintain the shape of the protective flavored thong 100.

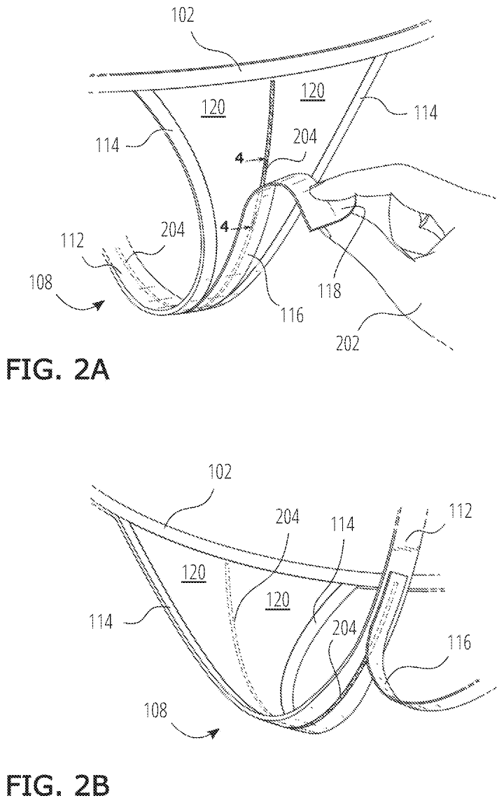

The protective flavored thong 100 includes a protective strip 116 that removably covers the flavor ridge 204 (see FIG. 1) before the thong 100 is used, e.g., worn to protect against disease transmission during cunnilingus. The protective strip 116 includes a tab portion 118 configured to facilitate removal of the protective strip 116 by pulling on the tab portion 118 using a wearer's hand 202 (shown in FIG. 2A).

With reference to FIG. 1B, an isometric rear view of the protective flavored thong 100 is shown, showing the waistband 102 including the front portion of the waistband 106 and the rear portion of the waistband 104. The crotch band 108 includes the wide end 110 and the narrow end 112. In this embodiment, the crotch band 108 includes two bands of elastic silicone 114. The protective flavored thong 100 includes the removable protective strip 116 that extends from the wide end 110 to the narrow end 112 of the crotch band 108, and protects the flavor ridge 204 (shown in FIG. 2A) until the protective flavored thong 100 is ready for use. In some embodiments, the wide end 110 of the crotch band 108 can include a panel 120 of low-density polyethylene (LDPE) that is impervious to fluids, thereby providing both the wearer and the wearer's partner (not shown) protection against sexually transmitted diseases (STDs).

With reference to FIG. 2A, an isometric close-up front view of the protective flavored thong 100 is shown, showing the waistband 102, the crotch band 108, and the two bands of elastic silicone 114. In this embodiment, the wide end 110 of the crotch band 108 includes a panel 120 of low-density polyethylene (LDPE). In this embodiment, the flavor ridge 204 is shown extending from the waistband 102 downward along the central axis of the panel 120, continuing along the central axis of the narrow end 112 of the crotch band 108.

In some embodiments, the flavor ridge 204 includes a flavoring agent and a sweetening agent (such as sucrose, agave, honey, or maple syrup). In some embodiments, the flavor ridge 204 includes a flavoring agent and a souring agent, such as citric acid (“sour salt”).

In some embodiments the flavor ridge 204 is flavored using a flavoring agent so as to resemble at least one of: lemon, lime, orange, cherry, pineapple. In some embodiments the flavor ridge 204 can be flavored to reflect a particular holiday, for example, candy corn flavor for Halloween, and candy cane flavor for Christmas. In some embodiments the flavor ridge 204 can be scented to mask unpleasant odors, and the flavor ridge 204 can be scented to enhance the erotic experience.

In some embodiments, the flavor ridge 204 forms a raised ridge above the surface of the crotch band 108 so as to provide lingual and tactile guidance in darkness while locating parts of the female anatomy generally under the central axis of the crotch band 108.

In some embodiments, a pair of flavor ridges 204 is applied symmetrically on each side of a central axis of the crotch band 108, the pair generally running along the central axis of the crotch band 108. In other embodiments, a trio of flavor ridges 204 is applied, the trio generally running along the central axis including two flavor ridges 204 symmetrically on each side of a central axis of the crotch band 108. Also shown is the wearer's hand 202 gripping the tab portion 118 of the protective strip 116, and pulling away the protective strip 116 to expose the flavor ridge 204.

In this embodiment, the protective strip 116 is configured to protect the freshness of the flavor ridge 204 while the protective strip 116 is attached to the flavor ridge 204, and the protective strip 116 can be removed so as to leave the flavor ridge 204 substantially in place after the protective strip 116 is removed.

In some embodiments, the protective flavored thong 100 is packaged in a folded condition (not shown). In some embodiments, the flavor ridge 204 folds once or twice when packaged in a folded condition.

With reference to FIG. 2B, an isometric close-up rear view of the protective flavored thong 100 is shown, showing the waistband 102, the crotch band 108, and the two bands of elastic silicone 114. The flavor ridge 204 is shown extending from the waistband 102 downward along the central axis of the panel 120, and continuing along the center of the narrow end 112 of the crotch band 108. Also shown is the protective strip 116 as it is almost completely removed from the crotch band 108. As the protective strip 116 is removed, the flavor ridge 204 is exposed.

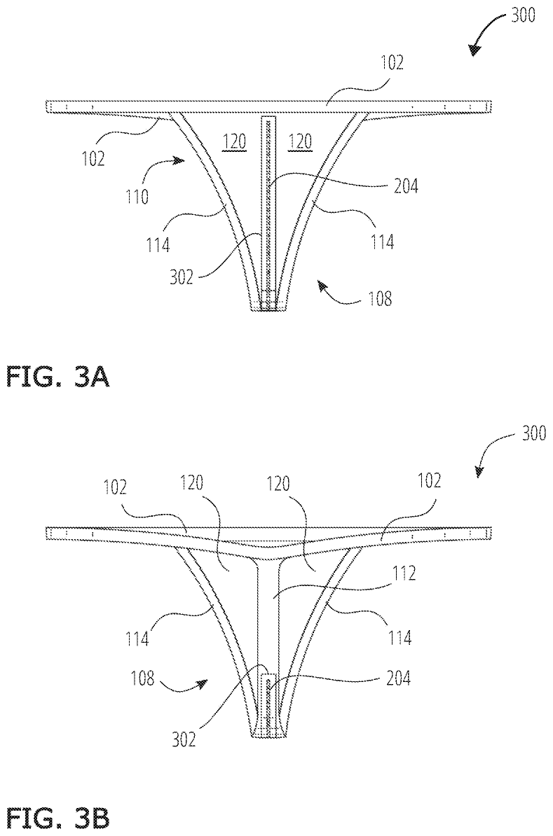

With reference to FIG. 3A, a front view of another embodiment 300 of a protective flavored thong 100 is shown, having a backing layer 302 that is applied along the central axis of the crotch band 108. The flavor ridge 204 can be pre-applied to the backing layer 302 before it is applied to the crotch band 108, or the flavor ridge 204 can be applied to the backing layer 302 after the backing layer 302 has been applied to the crotch band 108.

The protective strip 116 can be applied to the flavor ridge 204 before the backing layer 320 having the flavor ridge 204 is applied to the crotch band 108.

The backing layer 302 can include a light adhesive on its back surface that improves the adhesion of the backing layer 302 to the crotch band 108. In some embodiments, a front-facing surface of the backing layer 302 also provides a smooth surface that retains its shape while being licked so as to facilitate licking of the flavor ridge 204 off of the backing layer 302.

This embodiment 300 also includes the waistband 102, the crotch band 108, and the two bands of elastic silicone 114. The protective strip 116 (shown in FIG. 2A) has been completely removed from the panel 120 of the crotch band 108, thereby exposing the centrally located flavor ridge 204 on the backing layer 302, which is shown extending from the waistband 102 downward along the central axis of the panel 120, continuing along the central axis of the narrow end 112 of the crotch band 108.

With reference to FIG. 3B, an isometric close-up rear view of the protective flavored thong 300 is shown, showing the waistband 102, the crotch band 108, and the two bands of elastic silicone 114. The protective strip 116 (shown in FIG. 2A) has been completely removed from the crotch band 108. The crotch band 108, the backing layer 302, and the flavor ridge 204 thereon, are shown running along the central axis of the narrow end 112 of the crotch band 108.

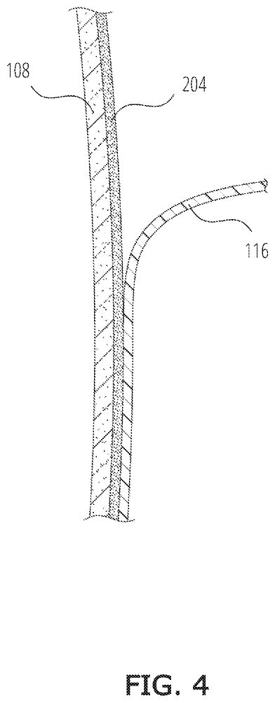

With reference to FIG. 4, a cross-sectional view of the layers of an embodiment of the protective flavored thong 100 is shown, showing the crotch band 108, the flavor ridge 204, and the protective strip 116. The protective strip 116 is shown being peeled away from the crotch band 108 and the flavor ridge 204. In some embodiments, the flavor ridge 204 includes at least one of: a flavoring agent, a sweetening agent, and a souring agent. In some embodiments the flavor ridge 204 is flavored to resemble one of: lemon, lime, orange, cherry, pineapple, grape, blueberry, mint, chocolate, licorice, cotton candy.

With reference to FIG. 5, a front view of the protective flavored thong 100 worn on the waist 504 of the wearer 502 is shown, showing the waistband 102, the two bands of elastic silicone 114, and the flavor ridge 204 running along the central axis of the panel 120.

Other modifications and implementations will occur to those skilled in the art without departing from the spirit and the scope of the invention as claimed. Accordingly, the above description is not intended to limit the invention, except as indicated in the following claims.

What is claimed is:

1. A protective flavored thong comprising:
a waistband configured to extend around a waist of a wearer, the waistband having a front portion and a rear portion;
a crotch band having a wide end and a narrow end, the narrow end being attached at the rear portion of the waistband, and the wide end being attached along the front portion of the waistband; and
at least one flavor ridge applied centrally along the crotch band.

2. The protective flavored thong of claim 1, wherein the waistband includes a band of elastic silicone.

3. The protective flavored thong of claim 1, wherein the crotch band includes two bands of elastic silicone, each band of elastic silicone running along an edge of the crotch band.

4. The protective flavored thong of claim 1, wherein the crotch band includes a panel of low-density polyethylene (LDPE).

5. The protective flavored thong of claim 1, wherein the crotch band includes a panel of material that is impervious to fluids.

6. The protective flavored thong of claim 1, wherein the at least one flavor ridge includes at least one of: a flavoring agent, a souring agent, and a sweetening agent.

7. The protective flavored thong of claim 1, further comprising:
a protective strip removably attached along the at least one flavor ridge, the protective strip being configured to protect freshness of the at least one flavor ridge while the at least one protective strip is attached to the at least one flavor ridge, and to be removable so as to leave the at least one flavor ridge substantially in place after the protective strip is removed.

8. The protective flavored thong of claim 7, wherein the protective strip includes a tab portion configured to facilitate removal of the protective strip.

9. The protective flavored thong of claim 1, wherein the at least one flavor ridge includes a pair of flavor ridges symmetrically placed along a central axis of the crotch band.

10. The protective flavored thong of claim 1, further comprising:
a backing layer, the backing layer applied centrally along the crotch band, the flavor ridge being applied centrally along the backing layer.

11. The protective flavored thong of claim 10, wherein the backing layer includes a coating of light adhesive that improves the adhesion of the backing layer to the crotch band.

12. The protective flavored thong of claim 10, wherein the backing layer includes a smooth front-facing surface that retains its shape so as to facilitate licking of the flavor ridge off of the backing layer.

13. The protective flavored thong of claim 1, wherein the protective flavored thong is packaged in a folded condition.

14. The protective flavored thong of claim 13, wherein the at least one flavor ridge folds once or twice when packaged in the folded condition.

15. The protective flavored thong of claim 1, wherein the at least one flavor ridge is flavored to resemble at least one of:
lemon, lime, orange, cherry, pineapple, grape, blueberry, licorice, chocolate, cotton candy.
